(19) United States
(12) Patent Application Publication
(10) Pub. No.: US 2011/0275679 A1
(43) Pub. Date: Nov. 10, 2011

(54) FLUPIRTINE HYDROCHLORIDE MALEIC ACID COCRYSTAL

(75) Inventors: Isabel Kalofonos, Cambridge, MA (US); Dimitris Kalofonos, Cambridge, MA (US); Patrick G. Stahly, West Lafayette, IN (US); Jeffrey S. Stults, West Lafayette, IN (US); Jason A. Hanko, West lafayette, IN (US); Rex A. Shipplett, Wolcott, IN (US); William Martin-Doyle, Cambridge, MA (US)

(73) Assignee: Bionevia Pharmaceuticals, Inc., Cambridge, MA (US)

(21) Appl. No.: 13/057,560

(22) PCT Filed: Aug. 6, 2009
(86) PCT No.: PCT/US09/52925

§ 371 Date: Jul. 26, 2011

Related U.S. Application Data
(60) Provisional application No. 61/086,644, filed on Aug. 6, 2008.

(51) Int. Cl. A61K 31/44; A61P 25/00; A61P 21/00; A61P 25/08; A61P 25/28; A61P 25/16; A61P 25/14; A61P 9/10; A61P 25/18; A61P 25/24; A61P 25/22; A61P 13/10; A61P 13/00; A61P 21/02; A61P 3/10; A61P 27/12; C07D 213/75

(52) U.S. Cl. 514/352; 546/308

(57) ABSTRACT

The invention relates to crystalline forms of flupirtine, particularly to 1:1 flupirtine hydrochloride maleic acid cocrystal. The preparation and characterization of 1:1 flupirtine hydrochloride maleic acid cocrystal is described. The invention also relates to the therapeutic use of the flupirtine hydrochloride maleic acid cocrystal to treat nervous system disorders, pain disorders, and musculoskeletal disorders and to pharmaceutical compositions containing the cocrystal.

### CROSS REFERENCE TO RELATED APPLICATIONS

This application claims priority to U.S. application Ser. No. 61/086,644, filed Aug. 6, 2008, which is incorporated herein by reference.

### FIELD OF THE INVENTION

The invention relates to crystalline forms of flupirtine, particularly to a 1:1 flupirtine hydrochloride maleic acid cocrystal. The invention also relates to its therapeutic use to treat nervous system disorders, pain disorders, and musculoskeletal disorders, and to pharmaceutical compositions containing the cocrystal.

### BACKGROUND OF THE INVENTION

Flupirtine, 2-amino-3-carbethoxyamino-6-(p-fluorobenzylamino)pyridine, (shown below) is a known active pharmaceutical ingredient (API) having beneficial analgesic, muscle relaxant, neuroprotective, and other nervous system activities and is useful in treating pain, muscle contracture-related, and other nervous system conditions.

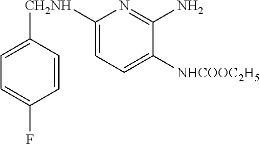

For example, flupirtine is therapeutically effective in the treatment of acute and chronic pain of various etiologies. Flupirtine also has positive indications for the treatment of neurodegenerative conditions. The preparation of flupirtine free base and a crystalline form of flupirtine hydrochloride salt are described in South African Pat. No. 69 02364, German Pat. No. 1795858; U.S. Pat. No. 3,481,943; U.S. Pat. No. 4,785,110; von Bebenburg W et al., Chemiker-Zeitung 1979; 103:387; and von Bebenburg W et al., Chemiker-Zeitung 1981; 105:217-219. The preparation and characterization of various crystalline forms of the flupirtine maleate salt are in German Pat. No. 31335191; U.S. Pat. No. 4,481,205; U.S. Pat. No. 5,959,115; WO2008/007117; Landgraf K F et al, European Journal of Pharmaceutics and Biopharmaceutics 46 (1998) 329-337; and Kuhnert-Brandstaetter M and Porsche U, Scientia Pharmaceutica 1990; 58; 55. The preparation of flupirtine gluconate is described in U.S. Pat. No. 4,673,666. The therapeutic activity of flupirtine maleate, which is the commercially available form, has been demonstrated in various conditions in the clinical literature, including but not limited to Bromm B et al., Postgrad Med J. 1987; 63 Suppl 3:109-12; Ceccarelli G et al., Postgrad Med J. 1987; 63 Suppl 3:105-8; Galasko C S et al., Curr Med Res Opin. 1985; 9:594-601; Göbel H et al., Schmerz. 1999 Oct. 15; 13(5):324-31; Goodchild C et al., Pain Medicine 2007; 8:612; Herrmann W M et al., Fortschr Med. 1993; 111:266-70; Herrmann W M et al., Postgrad Med J. 1987; 63 Suppl 3:87-103; Heusinger J H, Postgrad Med J. 1987; 63 Suppl 3:71-9; Lüben V et al., Fortschr Med. 1994; 112:282-6; Mastronardi P et al., J Int Med. Res. 1988; 16:338-48; McMahon F G et al., Postgrad Med J. 1987; 63 Suppl 3:81-5; Million R et al., Curr Med Res Opin. 1984; 9:204-12; Moore R A et al., Br J Anaesth. 1983; 55:429-32; Müller-Schwefe G, Fortschr Med. Orig. 2003; 121:11-18; Müller-Schwefe MMW Fortschr Med. 2004; 146 Spec No 2:76; Müller-Schwefe G H et al., MMW Fortschr Med. 2007 25; 149:153-161; Otto M et al., Neurology. 2004; 62:714-8; Riethmüller-Winzen H, Postgrad Med J. 1987; 63 Suppl 3:61-5; Ringe J D et al., Arzneimittelforschung. 2003; 53:496-502; Salembier L et al., Acta Otolaryngol Suppl. 2006; 556:93-5; Scheef W et al., Arzneimittelforschung. 1985; 35:75-7; Scheef W, Postgrad Med J. 1987; 63 Suppl 3:67-70; Stoll A L, Psychosomatics. 2000; 41:371-2; Wörz R et al., Fortschr Med. 1996; 114:500-4; and Wörz R et al., Fortschr Med. 1995; 113:463-8; and in the patent prior art, including U.S. Pat. No. 4,668,684; U.S. Pat. No. 4,778,799; U.S. Pat. No. 5,162,346; U.S. Pat. No. 5,284,861; U.S. Pat. No. 5,521,178; U.S. Pat. No. 5,721,258; U.S. Pat. No. 6,034,111; U.S. Pat. No. 6,034,112; U.S. Pat. No. 6,124,326; U.S. Pat. No. 6,610,324; U.S. Pat. No. 6,821,995; U.S. Pat. No. 7,309,713; WO/2002/015907; WO/2005/000306; WO/2005/058319; WO/2006/079559; WO/2007/128462.

Although therapeutic efficacy is the primary concern for a therapeutic agent, like flupirtine, the salt or solid state form (i.e., the crystalline or amorphous form) of a drug candidate can be critical to its pharmacological properties and to its development as a viable API. For example, each salt or each crystalline form of a drug candidate can have different solid state (physical and chemical) properties. The differences in physical properties exhibited by a novel solid form of an API (such as a cocrystal, salt, or polymorph of the original compound), affect pharmaceutical parameters such as storage stability, compressibility and density (important in formulation and product manufacturing), and solubility and dissolution rates (important factors in determining bioavailability). Because these practical properties are influenced by the solid state form of the API, they can significantly impact the selection of a compound as an active pharmaceutical ingredient (API), the ultimate pharmaceutical dosage form, the optimization of manufacturing processes, and absorption in the body. Moreover, finding the most adequate form for further drug development can reduce the time and the cost of that development.

Obtaining pure crystalline forms, then, is extremely useful in drug development. It permits better characterization of the drug candidate's chemical and physical properties. Crystalline forms often have better chemical and physical properties than amorphous forms. The crystalline form may possess more favorable pharmacology than the amorphous form or be easier to process. It may also have better storage stability.

One such physical property, which can affect processability, is the flowability of the solid, before and after milling. Flowability affects the ease with which the material is handled during processing into a pharmaceutical composition. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its dissolution rate in aqueous fluid. The rate of dissolution of an active ingredient in a patient's stomach fluid may have therapeutic consequences since it impacts the rate at which an orally administered active ingredient may reach the patient's bloodstream.

Another important solid state property of a pharmaceutical compound is its thermal behavior, including its melting point. The melting point of the solid form of a drug must be high enough to avoid melting or plastic deformation during standard processing operations, as well as concretion of the drug by plastic deformation on storage (Gould, P. L. Int. J. Pharmaceutics 1986 33 201-217). Normally a solid form should melt above about 100° C. to be considered optimum for development. For example, melting point categories used by one pharmaceutical company are, in order of preference, +(mp>120° C.), 0 (mp 80-120° C.), and −(mp<80° C.) (Balbach, S.; Korn, C. Int. J. Pharmaceutics 2004 275 1-12).

It is also possible to achieve desired properties of a particular API by forming a cocrystal of the API itself or of a salt of the API. Cocrystals are crystals that contain two or more non-identical molecules. Examples of cocrystals may be found in the Cambridge Structural Database. Examples of cocrystals may also be found at Etter, M. C., and Adsmond, D. A., J. Chem. Soc., Chem. Commun. 1990 589-591; Etter, M. C., MacDonald, J. C., and Bernstein, J., Acta Crystallogr., Sect. B, Struct. Sci. 1990 B46 256-262; and Etter, M. C., Urbańczyk-Lipkowska, Z., Zia-Ebrahimi, M., and Panunto, T. W., J. Am. Chem. Soc. 1990 112 8415-8426, which are incorporated herein by reference in their entireties. The following articles are also incorporated herein by reference in their entireties: Görbotz C. H., and Hersleth, H. P. Acta Cryst. 2000 B56 625-534; and Senthil Kumar, V. S., Nangia, A., Katz, A. K., and Carrell, H. L., Crystal Growth & Design, 2002 2 313-318.]

By cocrystallizing an API or a salt of an API with a co-former (the other component of the cocrystal), one creates a new solid state form of the API which has unique properties compared with existing solid forms of the API or its salt. For example, a cocrystal may have different dissolution and solubility properties than the active agent itself or its salt. Cocrystals containing APIs can be used to deliver APIs therapeutically. New drug formulations comprising cocrystals of APIs with pharmaceutically acceptable co-formers may have superior properties over existing drug formulations.

A crystalline form of a compound, a crystalline salt of the compound or a cocrystal containing the compound or its salt form generally possesses distinct crystallographic and spectroscopic properties when compared to other crystalline forms having the same chemical composition. Crystallographic and spectroscopic properties of the particular form are typically measured by X-ray powder diffraction (XRPD), single crystal X-ray crystallography, solid state NMR spectroscopy, e.g. ¹³C CP/MAS NMR, or Raman spectrometry, among other techniques. The particular crystalline form of a compound, of its salt, or of a cocrystal often also exhibit distinct thermal behavior. Thermal behavior is measured in the laboratory by such techniques as capillary melting point, thermogravimetric analysis (TGA) and differential scanning calorimetry (DSC).

As mentioned above, German Pat. No. 1795858; U.S. Pat. No. 3,481,943; U.S. Pat. No. 4,785,110; von Bebenburg W et al., Chemiker-Zeitung 1979; 103:387; and von Bebenburg W et al., Chemiker-Zeitung 1981; 105:217-219 describe the synthesis of a family of compounds including flupirtine free base and a crystalline form of flupirtine hydrochloride. German Pat. No. 31335191, U.S. Pat. No. 4,481,205, U.S. Pat. No. 5,959,115, and WO2008/007117 describe the synthesis and basic activities of flupirtine maleate, which is the available form for therapeutic use. Hlavica P et al., Arzneimittelforschung 1985; 35:67-74 describes the pharmacokinetic parameters of flupirtine in man, including a time to maximum plasma concentration (tₘₐₓ) of 2 hours. As described in Macheras P et al., Pharmaceutical Research 2000; 17:108-112, the dissolution rate of a compound can have effects on tₘₐₓ, and a drug with faster dissolution rate may also have a shorter tₘₐₓ. As described in Yüksel N, European Journal of Pharmaceutics and Biopharmaceutics 2003; 56:453-459, for drugs intended to be used as acute analgesics, a shorter tₘₐₓ is considered superior, since this results in a faster time to pain relief. Geisslinger G et al, Int J Clin Pharmacol Ther Toxicol 1989; 27:324-8 describe improving the tₘₐₓ and onset of action of ibuprofen free acid by administration of ibuprofen lysine salt, which has a faster dissolution rate than the free acid. Flupirtine maleate is reported to be insoluble in water (Sigma-Aldrich flupirtine maleate product information, 2008), and the dissolution rate of flupirtine maleate was measured at 0.088 [μg/mL]/min. A formulation of flupirtine with a higher dissolution rate than the flupirtine maleate salt may therefore have superior properties over existing drug formulations, particularly for use as an acute analgesic, for which a shorter tₘₐₓ is desirable.

Accordingly, there is a need in the art to increase the dissolution rate of flupirtine maleate. This invention answers those needs by providing a flupirtine hydrochloride maleic acid cocrystal with improved properties, specifically a faster dissolution rate. The invention also relates to processes of preparing the flupirtine hydrochloride maleic acid cocrystal, pharmaceutical compositions containing it, and its use to treat nervous system disorders, pain disorders, and musculoskeletal disorders.

### SUMMARY OF THE INVENTION

The invention relates to flupirtine hydrochloride maleic acid cocrystal, in particular a 1:1 flupirtine hydrochloride maleic acid cocrystal. This novel cocrystal exhibits an improved dissolution rate in comparison to the previously known flupirtine maleate.

### BRIEF DESCRIPTION OF THE FIGURES

FIG. 1-1 depicts the XRPD pattern of crystalline maleic acid form I.

FIG. 1-2 depicts the XRPD pattern of crystalline maleic acid form II.

FIG. 2-1 depicts the flupirtine maleate salt XRPD pattern.

FIG. 2-2 depicts the flupirtine maleate salt proton NMR spectrum.

FIG. 2-3 depicts the intrinsic dissolution curve (absorbance vs. time) for flupirtine maleate salt in water, measured by UV absorbance at 343 nm.

FIG. 2-4 depicts the intrinsic dissolution curve (concentration vs. time) for flupirtine maleate salt in water, measured by UV absorbance at 343 nm.

FIG. 5-1 depicts XRPD patterns of 1:1 flupirtine hydrochloride maleic acid cocrystal.

FIG. 5-2 depicts XRPD patterns of 1:1 flupirtine hydrochloride maleic acid cocrystal.

FIG. 5-3 depicts the proton NMR of 1:1 flupirtine hydrochloride maleic acid cocrystal.

FIG. 5-4 depicts the proton NMR of 1:1 flupirtine hydrochloride maleic acid cocrystal.

FIG. 5-5 depicts the DSC/TG analyses of 1:1 flupirtine hydrochloride maleic acid cocrystal.

FIG. 5-6 depicts the Raman spectrum of 1:1 flupirtine hydrochloride maleic acid cocrystal.

FIG. 5-7 depicts the dynamic vapor sorption plot of 1:1 flupirtine hydrochloride maleic acid cocrystal, measured by UV absorbance at 343 nm.

FIG. 5-8 depicts the intrinsic dissolution curve for 1:1 flupirtine hydrochloride maleic acid cocrystal, measured by UV absorbance at 343 nm.

FIG. 5-9 depicts an D overlay comparing 1:1 flupirtine hydrochloride maleic acid cocrystal (top) with four forms of crystalline flupirtine HCl salt (bottom four).

FIG. 5-10 depicts an XRPD overlay comparing 1:1 flupirtine hydrochloride maleic acid cocrystal (top) with crystalline maleic acid, form I and crystalline maleic acid, form II (bottom two). Also shown are four forms of crystalline flupirtine HCl salt.

FIG. 6-1 depicts the XRPD pattern of the 1:1 flupirtine hydrochloride maleic acid cocrystal from Example 6.3.

FIG. 6-2 depicts the proton NMR Spectrum of the 1:1 flupirtine hydrochloride maleic acid cocrystal from Example 6.3.

FIG. 6-3 depicts the DSC analysis of the flupirtine hydrochloride maleic acid cocrystal from Example 6.3.

FIG. 6-4 depicts the TGA analysis of the flupirtine hydrochloride maleic acid cocrystal from Example 6.3.

### DETAILED DESCRIPTION OF THE INVENTION

The invention relates to a flupirtine hydrochloride maleic acid cocrystal. The cocrystal of the invention exhibit improved properties, including faster dissolution rate, in comparison to that known for flupirtine maleate. The preparation and characterization of the flupirtine hydrochloride maleic acid cocrystal is described below in the examples.

Flupirtine hydrochloride maleic acid cocrystal was obtained in a crystalline solid form which is characterized by XRPD, Raman, and DVS. The formation of the 1:1 flupirtine hydrochloride maleic acid cocrystal is supported by its ¹H NMR spectrum. The XRPD patterns comparing the cocrystal with each known form of maleic acid and each known form of flupirtine hydrochloride confirm the formation of the cocrystal. The intrinsic dissolution data confirm that the flupirtine HCl maleic acid cocrystal has a faster dissolution rate than flupirtine maleate (0.21 vs. 0.088 [μg/mL]/min).

In its XRPD pattern, 1:1 flupirtine hydrochloride maleic acid cocrystal may be characterized by two or more peaks at 7.3°2θ±0.2°2θ; 8.6°2θ±0.2°2θ; 9.6°2θ±0.2°2θ; 10.8°2θ±0.2°2θ; 12.4°2θ±0.2°2θ; 13.7°2θ±0.2°2θ; and 16.2°2θ±0.2°2θ. For instance, 1:1 flupirtine hydrochloride maleic acid cocrystal may be characterized by peaks at 7.3°2θ±0.2°2θ; 8.6°2θ±0.2°2θ; and 10.8°2θ±0.2°2θ. Other peaks outside this list, such as any of those listed in Tables 5-1, 5-2 and 6-1 below, may also be used for purposes of characterizing the 1:1 flupirtine hydrochloride maleic acid cocrystal. The 1:1 flupirtine hydrochloride maleic acid cocrystal may also be characterized by its Raman spectra, described below, and by combinations of two or more peaks shown in the Raman spectrum.

### Pharmaceutical Compositions and Methods of Treatment

The flupirtine hydrochloride maleic acid cocrystal of the invention possesses the same pharmacological activity as flupirtine free base and its salts, such as flupirtine maleate, and is useful for treating nervous system disorders, pain disorders, and musculoskeletal conditions such as those discussed above, especially acute and chronic pain of various etiologies, including back pain, neck pain, pain resulting from traumatic injury, post-operative pain, post-dental procedure pain, dysmenorrhea, osteoarthritis, visceral pain, cancer pain, rheumatoid arthritis, psoriatic arthritis, gout, tendonitis pain, bursitis pain, musculoskeletal pain, sports injury-related pain, sprains, strains, pain of osteoporosis, ankylosing spondylitis, headache of various etiologies including but not limited to migraine and tension headache, temporomandibular joint pain, fibromyalgia, myofascial pain syndrome, pain of irritable bowel syndrome, interstitial cystitis, and idiopathic chronic pain.

The flupirtine hydrochloride maleic acid cocrystal of the invention is also useful for treating acute and chronic neuropathic pain, and pain associated with nervous system disorders, including but not limited to, painful diabetic neuropathy, postherpetic neuralgia, trigeminal neuralgia, complex regional pain syndrome I, complex regional pain syndrome II, ischemic neuropathy, phantom limb pain, chemotherapy-induced neuropathy, HIV-related neuropathy, AIDS-related neuropathy, neuropathic back pain, neuropathic neck pain, carpal tunnel syndrome, other forms of nerve entrapment or nerve compression pain, brachial plexus lesions, other peripheral nerve lesions, neuropathic cancer pain, vulvodynia, central neuropathic pain, pain due to multiple sclerosis, post-stroke pain, Parkinson's Disease related central pain, postoperative chronic pain, Guillain-Barre syndrome (GBS), Charcot-Marie-Tooth (CMT) disease, idiopathic peripheral neuropathy, alcoholic neuropathy, other types of neuropathic pain, and other nervous system disorders that have pain as an attendant sign and/or symptom.

The flupirtine hydrochloride maleic acid cocrystal of the invention exerts a muscle relaxant effect, and is also useful for treating acute and chronic conditions of pathological muscle contracture, including but not limited to the discomfort, muscle spasm, stiffness, or myotonic conditions associated with painful musculoskeletal conditions, such as back pain, neck pain, neck-shoulder-arm syndrome, scapulohumeral periarthritis, cervical spondylosis, and other musculoskeletal conditions; spasticity or spastic paralysis of neurological origin due to multiple sclerosis, spinal cord injury, traumatic brain injury, cerebral palsy, stroke or cerebrovascular disorder, spastic spinal paralysis, sequelae of surgical trauma (including cerebrospinal tumor), amyotrophic lateral sclerosis, spinocerebellar degeneration, spinal vascular disorders, subacute myelo-optico neuropathy (SMON) and other encephalomyelopathies, and other neurological conditions; primary dystonia; secondary dystonia; and muscle cramps.

The flupirtine hydrochloride maleic acid cocrystal of the invention has nervous system activity and neuroprotective effects, and is also useful for treating a variety of nervous system conditions including, but not limited to epilepsy, Creutzfeldt-Jakob Disease, Alzheimer's Disease, Parkinson's Disease, Huntington's Disease, Batten Disease, cerebral ischemia, schizophrenia, psychosis, mood disorders including bipolar disorder, major depressive disorder, dysthymia, anxiety disorders, overactive bladder, urinary incontinence, urinary flow problems as a result of prostate hyperplasia, irritable bowel syndrome, and tinnitus

The flupirtine hydrochloride maleic acid cocrystal of the invention is also useful for treating diabetes mellitus and neurodegenerative diseases of the nervous and visual systems resulting as a complication of diabetes, including but not limited to diabetic neuropathy, diabetic retinopathy, diabetic maculopathy, glaucoma, diabetic gastroparesis, cataracts, and foot ulcers; for preventing and treating diseases associated with an impairment of the hematopoietic cell system, including but not limited to HIV and AIDS; for preventing and treating disorders which are associated with an unphysiologically high cell death rate, including but not limited to organ disorders resulting from myocardial infarct, cardiogenic shock, kidney shock, lung shock, and to other disorders associated with a high cell death rate including but not limited to senile macular degeneration and traumas resulting from mechanical, thermal, radiation, or other toxic influences.

The flupirtine hydrochloride maleic acid cocrystal of the invention is also useful for administration in combination with other analgesic medication classes, such as strong and weak opioids, NSAIDs, COX-2 inhibitors, acetaminophen, other anti-inflammatories, tricyclic antidepressants, anticonvulsant agents, voltage gated calcium channel blockers, N-type calcium channel blockers, other calcium channel modulators, SNRIs and other monoamine reuptake inhibitors, sodium channel blockers, NMDA antagonists, AMPA antagonists, other glutamate modulators, GABA modulators, CRMP-2 modulators, NK-1 antagonists, TRPV1 agonists, cannabinoids, adenosine agonists, nicotinic agonists, p38 MAP kinase inhibitors, corticosteroids, and other analgesic drug classes, and might have a useful dose-sparing effect of lowering the required dosage of the medication used in combination with the flupirtine hydrochloride maleic acid cocrystal of the invention. The flupirtine hydrochloride maleic acid cocrystal of the invention is therefore also useful for treating or preventing complications or side effects arising from usage of other analgesic medications, including problems with opioids such as dependency, constipation, and respiratory depression. Opioid pain medications can either inhibit or excite the CNS, although it is considered that inhibition is more common. Patients with depressed CNS functions may feel varying levels of drowsiness, lightheadedness, euphoria or dysphoria, or confusion. NSAID pain medications can also induce negative side effects, such as gastrointestinal toxicity or bleeding, renal toxicity, and cardiovascular toxicity. Side effects of other analgesic classes can include sedation, dizziness, anticholinergic effects, dependency, hypotension, and various other adverse effects. These analgesic-induced side effects can manifest themselves when the dosage is increased. Decreasing the dosage or changing medications often helps to decrease the rate or severity of these analgesic-induced side effects. It is possible that a therapeutic amount of the flupirtine hydrochloride maleic acid cocrystal of the invention in combination with a pain agent will reduce the risk of such side effects by reducing the required dosage of the other agent used in combination.

As discussed, the invention relates to pharmaceutical compositions comprising a therapeutically effective amount of the flupirtine hydrochloride maleic acid cocrystal of the invention and a pharmaceutically acceptable carrier (also known as a pharmaceutically acceptable excipient). The flupirtine hydrochloride maleic acid cocrystal of the invention has the same pharmaceutical activity as previously reported for flupirtine and its salts, such as flupirtine maleate. Pharmaceutical compositions for the treatment of those conditions or disorders contain a therapeutically effective amount of the flupirtine hydrochloride maleic acid cocrystal of the invention, as appropriate, for treatment of a patient with the particular condition or disorder. A “therapeutically effective amount” of the flupirtine hydrochloride maleic acid cocrystal, according to the invention (discussed here concerning the pharmaceutical compositions), refers to an amount of a therapeutic agent to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The actual amount required for treatment of any particular patient will depend upon a variety of factors including the disorder being treated and its severity; the specific pharmaceutical composition employed; the age, body weight, general health, sex and diet of the patient; the mode of administration; the time of administration; the route of administration; and the rate of excretion of flupirtine; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001, which is incorporated herein by reference.

A pharmaceutical composition of the invention may be any pharmaceutical form which contains the flupirtine hydrochloride maleic acid cocrystal of the invention. Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition of the invention, that is one having the flupirtine hydrochloride maleic acid cocrystal of the invention, a carrier should be chosen that maintains its crystalline form. In other words, the carrier should not substantially alter the crystalline form of the flupirtine hydrochloride maleic acid cocrystal of the invention. Nor should the carrier be otherwise incompatible with flupirtine itself or the flupirtine hydrochloride maleic acid cocrystal of the invention, such as by producing any undesirable biological effect or otherwise interacting in a deleterious manner with any other component(s) of the pharmaceutical composition.

The pharmaceutical compositions of the invention are preferably formulated in unit dosage form for ease of administration and uniformity of dosage. A “unit dosage form” refers to a physically discrete unit of therapeutic agent appropriate for the patient to be treated. It will be understood, however, that the total daily dosage of the flupirtine hydrochloride maleic acid cocrystal of the invention and its pharmaceutical compositions according to the invention will be decided by the attending physician within the scope of sound medical judgment.

Because the flupirtine hydrochloride maleic acid cocrystal of the invention exists in a crystalline form, solid dosage forms are a preferred form for the pharmaceutical composition of the invention. Solid dosage forms for oral administration include capsules, tablets, pills, powders, and granules. Tablets are particularly preferred. The active ingredient may be contained in a solid dosage form formulation that provides quick release, sustained release or delayed release after administration to the patient. In such solid dosage forms, the active compound is mixed with at least one inert, pharmaceutically acceptable carrier such as sodium citrate or dicalcium phosphate. The solid dosage form may also include one or more of: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar, calcium carbonate, potato or tapioca starch, alginic acid, certain silicates, and sodium carbonate; e) dissolution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, and sodium lauryl sulfate. The solid dosage forms may also comprise buffering agents. They may optionally contain opacifying agents and can also be of a composition that they release the active ingredient(s) only, or preferentially, in a certain part of the intestinal tract, optionally, in a delayed manner. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Solid dosage forms of pharmaceutical compositions of the invention can also be prepared with coatings and shells such as enteric coatings and other coatings well known in the pharmaceutical formulating art.

The flupirtine hydrochloride maleic acid cocrystal of the invention can be in a solid micro-encapsulated form with one or more carriers as discussed above. Microencapsulated forms may also be used in soft and hard-filled gelatin capsules with carriers such as lactose or milk sugar as well as high molecular weight polyethylene glycols and the like.

The flupirtine hydrochloride maleic acid cocrystal may also be used in the preparation of non-solid formulations, e.g., injectables and patches, of flupirtine. Such non-solid formulations are known in the art. In anon-solid formulation, the crystalline form is, generally speaking, not maintained. For example, the crystalline form may be dissolved in a liquid carrier. In this case, the crystalline forms of the invention represent intermediate forms of flupirtine used in the preparation of the non-solid formulation. The crystalline forms of the invention provide advantages of handling stability and purity to the process of making such formulations.

The invention also relates to the treatment of nervous system disorders, pain disorders, and musculoskeletal disorders such as those discussed above. The invention provides a method for treating of nervous system disorders, pain disorders, and musculoskeletal disorders by administering to mammals the flupirtine hydrochloride maleic acid cocrystal of the invention, or a pharmaceutical composition containing it, in an amount sufficient to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The cocrystal and pharmaceutical compositions containing it, according to the invention, may be administered using any amount, any form of pharmaceutical composition and any route of administration effective for the treatment. After formulation with an appropriate pharmaceutically acceptable carrier in a desired dosage, as known by those of skill in the art, the pharmaceutical compositions of this invention can be administered to humans and other animals orally, rectally, or topically (as by powders or other solid form-based topical formulations). In certain embodiments, the flupirtine hydrochloride maleic acid cocrystal of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. As discussed above, the amount required for treatment of a particular patient will depend upon a variety of factors including the disorder being treated and its severity; the specific pharmaceutical composition employed; the age, body weight, general health, sex and diet of the patient; the mode of administration; the time of administration; the route of administration; and the rate of excretion of flupirtine; the duration of the treatment; any drugs used in combination or coincidental with the specific compound employed; and other such factors well known in the medical arts. And, as also discussed, the pharmaceutical composition of the flupirtine hydrochloride maleic acid cocrystal may be administered as a unit dosage form.

### EXAMPLES

Example 1 describes the characterization of maleic acid. Example 2 describes the characterization of flupirtine maleate. Example 3 describes the preparation of flupirtine free base. Example 4 describes the preparation of flupirtine hydrochloride. Example 5 describes the preparation and characterization of the 1:1 flupirtine hydrochloride maleic acid cocrystal. In Examples 3-5, three different samples, samples (a), (b), and (c), have been prepared for each of the flupirtine free base, flupirtine hydrochloride, and cocrystal preparations. Example 6 describes the preparation of the 1:1 flupirtine hydrochloride maleic acid cocrystal using a milling technique. The following methods and instruments were used to characterize these crystalline forms.

One of skill in the art would appreciate that certain analytical techniques, such as, for example, XRPD, ¹H-NMR, DSC, TGA, and Raman, will not produce exactly the same results every time due to, for example, instrumental variation, sample preparation, scientific error, etc. By way of example only, XRPD results (i.e. peak locations, intensities, and/or presence) may vary slightly from sample to sample, despite the fact that the samples are, within accepted scientific principles, the same form, and this may be due to, for example, preferred orientation. It is well within the ability of those skilled in the art, looking at the data as a whole, to appreciate whether such differences indicate a different form, and thus determine whether analytical data being compared to those disclosed herein are substantially similar. In this regard, and as is commonly practiced within the scientific community, it is not intended that the exemplary analytical data of the 1:1 flupirtine hydrochloride maleic acid cocrystal according to the invention disclosed here be met literally in order to determine whether comparative data represent the same form as those disclosed and claimed herein, such as, for example, whether each and every peak of an exemplary XRPD pattern in comparative data, in the same location, and/or of the same intensity. Rather it is intended that those of skill in the art, using accepted scientific principles, will make a determination regarding whether comparative analytical data represent the same or a different form.

X-Ray Powder Diffraction (XRPD):

Shimadzu XRD-6000 Diffractometer: Samples were analyzed using a Shimadzu XRD-6000 X-ray powder diffractometer using Cu Kα radiation. The instrument is equipped with a long fine focus X-ray tube. The tube voltage and amperage were set at 40 kV and 40 mA, respectively. The divergence and scattering slits were set at 1° and the receiving slit was set at 0.15 mm. Diffracted radiation was detected by a NaI scintillation detector. A theta-two theta continuous scan at 3°/min (0.4 sec/0.02° step) from 2.5 to 40°2θ was used. A silicon standard was analyzed to check the instrument alignment. Samples were prepared for analysis by placing them in an aluminum/silicon sample holder.

Inel XRG-3000 Diffractometer: X-ray powder diffraction (XRPD) analyses were performed using an Inel XRG-3000 diffractometer equipped with a CPS (Curved Position Sensitive) detector with a 2θ range of 120°. Real time data were collected using Cu-Kα radiation. The tube voltage and amperage were set to 40 kV and 30 mA, respectively. The monochromator slit was set at 1-5 mm by 160 μm. The patterns are displayed from 2.5-40°2θ). Samples were prepared for analysis by packing them into thin-walled glass capillaries. Each capillary was mounted onto a goniometer head that is motorized to permit spinning of the capillary during data acquisition. The samples were analyzed for 300 seconds. Instrument calibration was performed using a silicon reference standard.

XRPD patterns were collected using a PANalytical X'Pert Pro diffractometer. An incident beam of Cu Kα radiation was produced using an Optix long, fine-focus source. An elliptically graded multilayer mirror was used to focus the Cu Kα X-rays of the source through the specimen and onto the detector. Data were collected and analyzed using X'Pert Pro Data Collector software (v. 2.2b). Prior to the analysis, a silicon specimen (NIST SRM 640c) was analyzed to verify the Si 111 peak position. The specimen was sandwiched between 3 μm thick films, analyzed in transmission geometry, and rotated to optimize orientation statistics. A beam-stop was used to minimize the background generated by air scattering. Soller slits were used for the incident and diffracted beams to minimize axial divergence. Diffraction patterns were collected using a scanning position-sensitive detector (X'Celerator) located 240 mm from the specimen.

Solution State ¹H NMR: The spectra were obtained on an INOVA-400 spectrometer. Samples were prepared for H NMR spectroscopy as ˜5-50 mg solutions in either CD₃OD or DMSO-d₆. Spectra were obtained using standard acquisition parameters.

Differential Scanning Calorimetry: Differential scanning calorimetry (DSC) was performed using a TA Instruments differential scanning calorimeter 2920. Each sample was placed in an aluminum DSC pan, and the weight accurately recorded. The pan was covered with a lid, then crimped and analyzed up to a final temperature of 250° C. Indium metal was used as the calibration standard. Reported temperatures are at the transition maxima.

Thermogravimetric analysis: Thermogravimetric (TG) analyses were performed using a TA Instruments 2950 thermogravimetric analyzer. Each sample was placed in an aluminum sample pan and inserted into the TG furnace. The furnace was first equilibrated at 25° C., then heated under nitrogen at a rate of 10° C./min, up to a final temperature of either 300 or 350° C. Nickel and Alumel™ were used as the calibration standards.

Dispersive Raman: FT Dispersive Raman spectra were acquired on a Renishaw Mk1 Ramascope model 1000 equipped with a Leica DM LM microscope. A 5× objective was used for the analysis. The excitation wavelength was 785 nm and the laser was at 10% power. A continuous grating scan from 3200 to 100 cm⁻¹ was used with an exposure time of 10 seconds and high gain. The samples were analyzed at a spectral resolution of 4.000. The samples were prepared for analysis by placing particles onto a gold mirror. The instrument was calibrated with a silicon wafer standard and a neon emission lamp.

Dynamic Vapor Sorption/Desorption (DVS): Moisture sorption/desorption data were collected on a VTI SGA-100 Vapor Sorption Analyzer. Sorption and desorption data were collected over a range of 5% to 95% relative humidity (RH) at 10% RH intervals under a nitrogen purge. Samples were not dried prior to analysis. Equilibrium criteria used for analysis were less than 0.0100% weight change in 5 minutes, with a maximum equilibration time of 3 hours if the weight criterion was not met. Data were not corrected for the initial moisture content of the samples. Sodium chloride and polyvinylpyrrolidine were used as calibration standards.

Equilibrium Solubility—UV Measurement: Equilibrium solubility was determined in water for the flupirtine HCl maleic acid cocrystal using ambient-temperature slurry experiments. Samples were prepared with excess solids and agitated on a wheel for at least 3 days. Suspended solids were removed by filtration. Concentrations were determined using ultraviolet spectrophotometry. Retained solids were analyzed by X-ray powder diffraction, if sufficient solids were present. Concentrations were calculated from the Beer's Law plots generated from the UV absorption of the aqueous standards for each material. A wavelength of approximately 342 nm was chosen for the cocrystal to avoid potential interference from maleic acid.

Ultraviolet spectrophotometry: Solutions were analyzed using a Cary 50 dual-beam spectrophotometer. They were analyzed at ambient temperature in a 1.000-cm quartz cuvette. Scans at 600 nm/min in the range of 800-200 nm were performed to determine an optimal wavelength for concentration measurement. The cuvette was washed with methanol, followed by water, and the detector was then zeroed prior to analysis of each sample. Wavelength calibration was performed using holmium oxide. The photometric accuracy was verified by measuring the intensity of the light at the detector when filters of known optical density were placed in the path of the beam.

Intrinsic Dissolution: Pellets of approximately 200 mg were pressed at 3000 lbs. for 1 minute in a standard Woods apparatus, with a surface area of 0.5 cm². One pellet was tested for each material. The samples were rotated in a VanKel dissolution apparatus, with automated sampling, at 100 RPM in 900 mL of water at 37° C. Aliquots were taken every two minutes and not filtered prior to analysis. Concentrations were calculated from the Beer's Law plots generated from the UV absorption of the aqueous standards for each material; however, the maleate salt plot was used for the cocrystal since the recovered solids exhibited a maleate salt XRPD pattern. A wavelength of approximately 343 nm was chosen for the cocrystal to avoid potential interference from maleic acid. To determine the rate of dissolution of each material, a plot was generated of the absorbance over time for each material. From this plot, a linear region was chosen from the initial dissolution period of each material. Concentrations were plotted versus time for the regions. A straight-line was fit to the data for each material. The slope of these lines provides the dissolution rate for each of the materials, expressed as [μg/mL]/min. The rates were not normalized for the surface area of the pellet.

Aliquots of the dissolution medium were removed manually at the end of the run and analyzed by UV spectrophotometry, and the remainder of the pellets were recovered for XRPD analysis.

### Example 1

### Characterization of Crystalline Maleic Acid

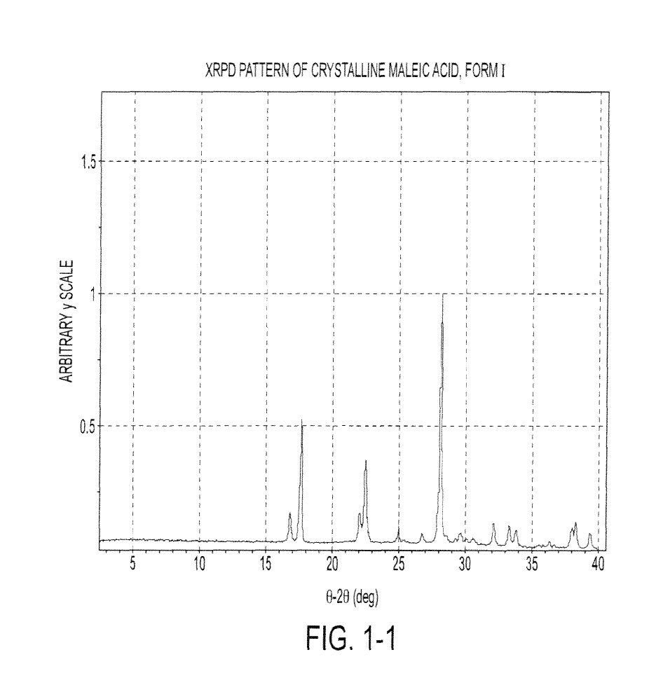

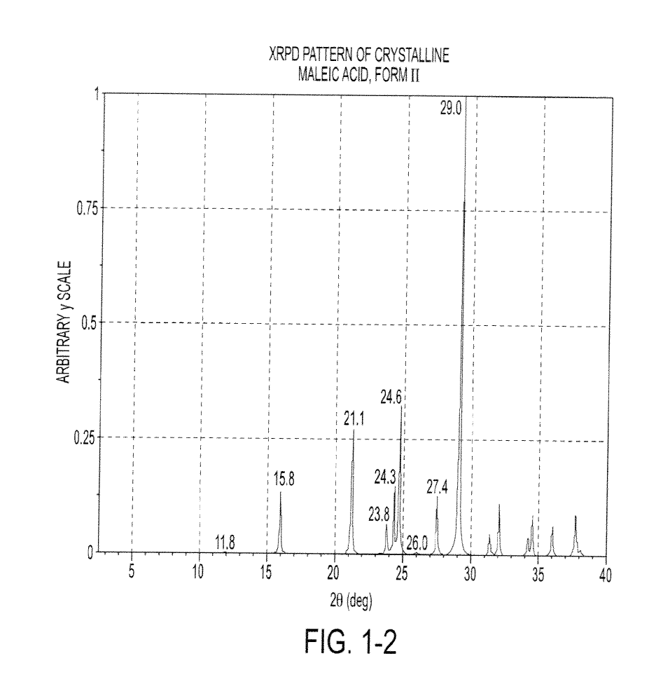

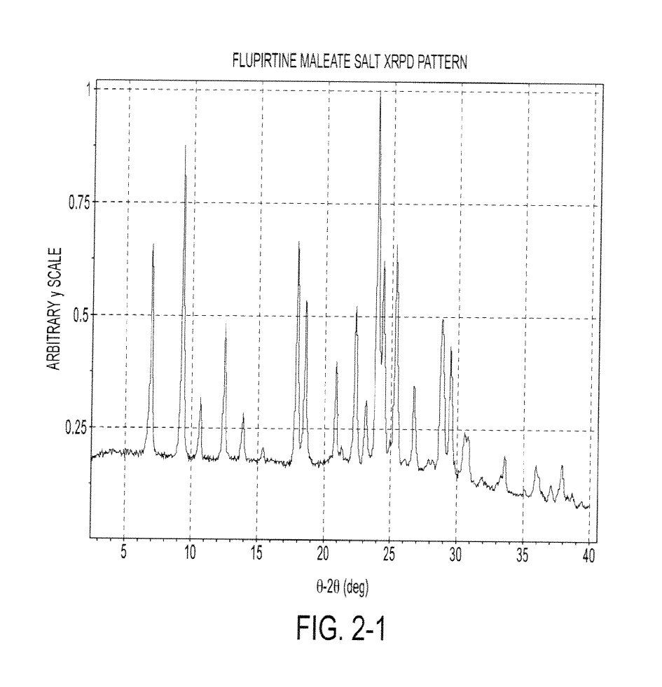

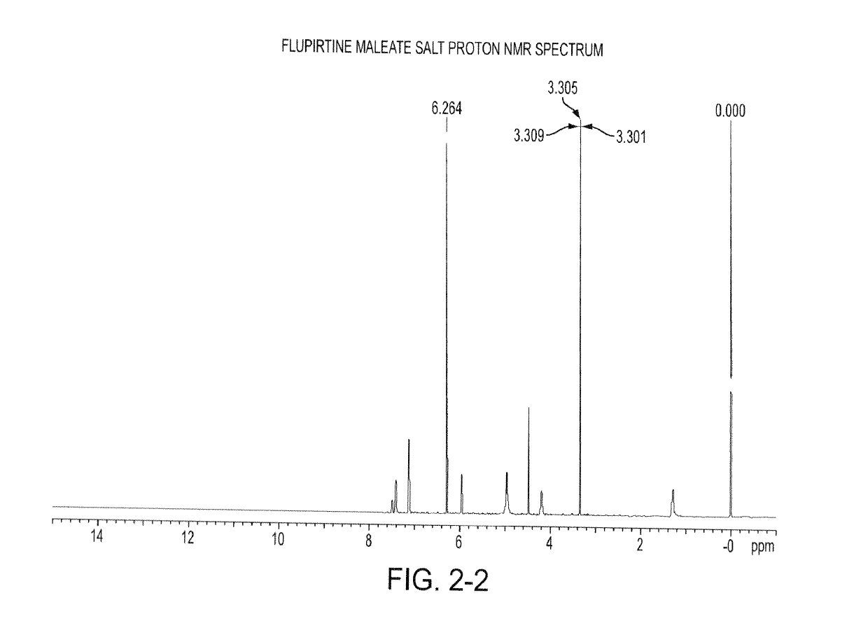

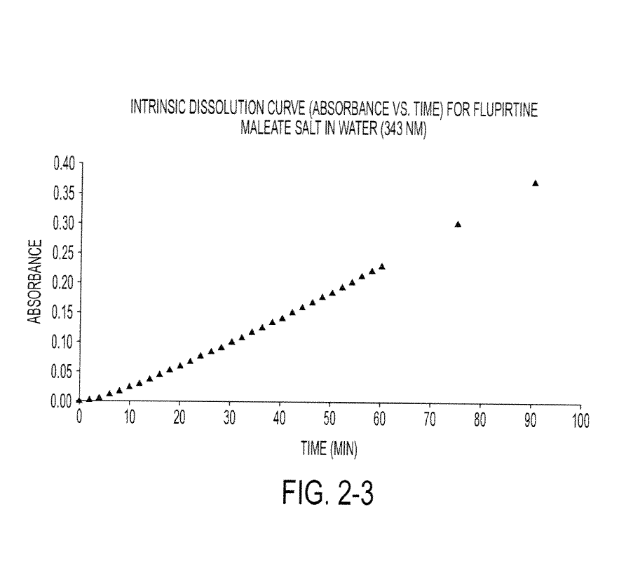

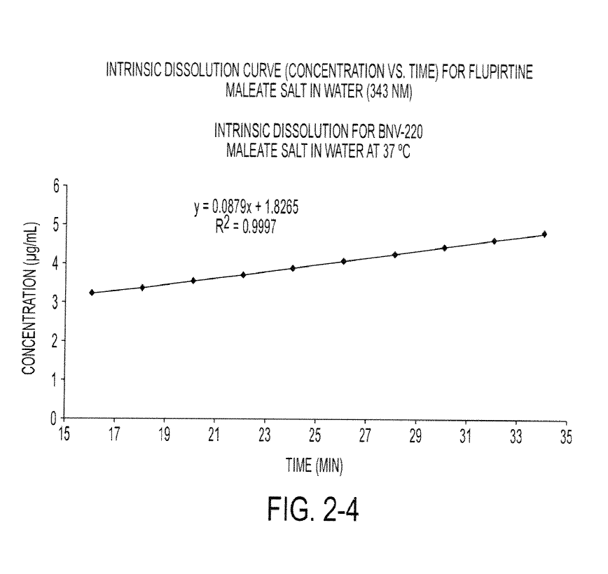

Crystalline maleic acid is available as two known forms, Form I and Form II. The XPRD data for both forms was obtained from the Cambridge Structural Database (Cambridge Crystallographic Data Centre). FIG. 1-1 is a representative XRPD pattern of crystalline maleic acid, Form I, and Table 1-1 reports the calculated peak positions the XRPD pattern for Form I. FIG. 1-2 is a representative XRPD pattern of crystalline maleic acid, Form II, and Table 1-2 reports the calculated peak positions in the XRPD pattern for Form II.

TABLE 1-1
Peak Positions of the XRPD Pattern for Maleic Acid, Form I

| °2θ | Intensity (%) |
|---|---|
| 16.7 | 13 |
| 17.6 | 29 |
| 22.1 | 12 |
| 22.5 | 22 |
| 22.7 | 7 |
| 25.0 | 3 |
| 25.5 | 1 |
| 26.8 | 4 |
| 28.1 | 100 |
| 28.7 | 4 |
| 29.4 | 3 |
| 29.7 | 4 |

TABLE 1-2
Peak Positions of the XRPD Pattern for Maleic Acid, Form II

| °2θ | Intensity (%) |
|---|---|
| 11.8 | 1 |
| 15.8 | 13 |
| 21.1 | 27 |
| 23.8 | 7 |
| 24.3 | 15 |
| 24.6 | 33 |
| 26.0 | 1 |
| 27.4 | 13 |
| 29.0 | 100 |

### Example 2

### Characterization of Flupirtine Maleate

Flupirtine maleate was obtained from Hallochem Pharma, Chonqing, China. Flupirtine maleate was characterized by XRPD. The XRPD pattern is shown in FIG. 2-1. Table 2-1 reports the peaks identified in the XRPD pattern.

TABLE 2-1
Peak Positions of the XRPD Pattern for Flupirtine Maleate

| | Intensity |
|---|---|
| degrees 2θ | % (I/Io) |
| 6.9 ± 0.2 | 57 |
| 9.3 ± 0.2 | 86 |
| 10.6 ± 0.2 | 17 |
| 12.5 ± 0.2 | 38 |
| 13.9 ± 0.2 | 13 |
| 15.4 ± 0.2 | 4 |
| 17.9 ± 0.2 | 60 |
| 18.5 ± 0.2 | 45 |
| 20.8 ± 0.2 | 27 |
| 21.3 ± 0.2 | 5 |
| 22.4 ± 0.2 | 43 |
| 23.2 ± 0.2 | 17 |
| 24.0 ± 0.2 | 100 |
| 24.4 ± 0.2 | 55 |
| 25.4 ± 0.2 | 60 |
| 26.8 ± 0.2 | 22 |
| 27.9 ± 0.2 | 4 |
| 28.9 ± 0.2 | 43 |
| 29.6 ± 0.2 | 35 |

Solution ¹H-NMR analysis, conducted in deuterated methanol, is shown in FIG. 2-2 and the peaks are listed in Table 2-2. The proton NMR confirms the chemical identity of flupirtine maleate.

TABLE 2-2
¹H-NMR Peak Positions

| | peak | | coupling | |
|---|---|---|---|---|
| | position | | constant | number |
| protons | (ppm) | multiplicity | (Hz) | of protons |
| CH₃ | 1.29 | triplet | 7 | 3 |
| CH₂O | 4.16 | quartet | 7 | 2 |
| CH₂N | 4.45 | singlet | — | 2 |
| aromatic | 5.94 | doublet | 9 | 1 |
| CH═CH (maleate) | 6.26 | singlet | — | 2 |
| aromatic | 7.07-7.12 | multiplet | — | 2 |
| aromatic | 7.37-7.40 | multiplet | — | 2 |
| aromatic | 7.46 | doublet | 9 | 1 |

FIG. 2-3 depicts the UV absorbance (at 343 nm) vs. time curves for the intrinsic dissolution experiment on flupirtine maleate in water at 37° C. The maleate salt exhibited low absorbance values, indicating poor aqueous solubility. FIG. 2-4 shows the portion of the data within the linear range of the Beer's Law plot in terms of concentration to determine the intrinsic dissolution rate of ˜0.088 [μg/mL]/min.

### Example 3a

### Preparation of Flupirtine Free Base

Diethyl ether (110 mL) and 3.01 g (7.16 mmol) of the flupirtine maleate of Example 2 were placed in a 250-mL separatory funnel. Saturated aqueous sodium bicarbonate (82 mL) was added and the funnel was shaken vigorously. The top, ether layer was removed, washed with three 11-mL portions of water. The spent wash was returned to the funnel, back extracting with diethyl ether (2×10 mL). The extracts were combined and dried over magnesium sulfate. The supernatant was filtered through a VWR 0.22-μm Teflon disc into a clean Erlenmeyer flask, rinsing the with three 10-mL portions of diethyl ether. Each portion was filtered and the filtrate added to the Erlenmeyer flask, to give a total volume of 127 mL of diethyl ether solution. A 64-mL portion of the solution was removed and allowed to evaporate overnight at ambient temperature under a blanket of nitrogen to give 1.05 g (3.45 mmol, 96% yield based on 64/127 of the starting material) of solid flupirtine free base.

### Example 3b

### Preparation of Flupirtine Free Base

Diethyl ether (50 mL) and 2.74 g (6.52 mmol) of the flupirtine maleate salt of Example 2 were placed in a 250-mL separatory funnel. Saturated aqueous sodium bicarbonate (50 mL) was added, and the funnel was shaken vigorously. Additional diethyl ether (50 mL) and saturated aqueous sodium bicarbonate (25 mL) were added with shaking, until no more solids were present. The top, ether layer was removed and washed with two 10-mL portions of water. The spent wash was returned to the funnel, back extracting with diethyl ether (2×10 mL) The extracts were combined and dried over magnesium sulfate. The mixture was filtered through qualitative filter paper, rinsing the spent drying agent three times with diethyl ether. A portion of the ether evaporated, resulting in precipitation of off-white solids in the filtrate. These solids were redissolved through sonication, and the resulting solution was filtered through a 0.22-μm Teflon disc directly into a 250-mL round-bottom flask. The ether was allowed to evaporate at ambient temperature under a blanket of nitrogen. Evaporation was completed through rotary evaporation (27-37° C., ˜30 in. Hg vacuum) to a constant weight, indicated by less than 0.01% weight loss between weighings, to give 2.03 g (6.67 mmol, 102% yield) of flupirtine free base as a solid.

### Example 3c

### Preparation of Flupirtine Free Base

A solution of 3.8 g (9.0 mmol) of flupirtine maleate from Example 2 in 1.8 L of water was prepared in a 3-L round-bottom flask by warming with a heating mantle. When the solution was at about 53° C. it was treated with 0.66 ml of 50% NaOH (12 mmol of NaOH), resulting in precipitation of solid. The heater was turned off and the mixture was gently stirred for approximately 17 hours. The mixture was vacuum filtered and the filter cake was washed with five 20-mL portions of water which were first used to rinse the flask. The solid was left on the filter under vacuum for about 30 minutes to dry, giving 2.5 g (91% yield) of solid flupirtine free base.

### Example 4a

### Preparation of Hydrogen Chloride Salt of Flupirtine (Flupirtine Hydrochloride)

The flupirtine free base (1.05 g, 3.45 mmol) of Example 3a was dissolved in 53 mL of diethyl ether with the aid of sonication. The solution was stirred under a blanket of nitrogen gas and treated drop wise, over approximately 2 minutes, with 0.300 mL of 37% hydrochloric acid (3.65 mmol of HCl) dissolved in 0.300 mL of diethyl ether. Precipitation occurred gradually. The resulting slurry was stirred overnight and filtered through qualitative filter paper. The wet solids were dried in a vacuum oven (23° C., ˜30 in. Hg vacuum) for approximately 15 hours to give 0.84 g (2.5 mmol, 71% yield) of solid flupirtine HCl.

### Example 4b

### Preparation of Flupirtine Hydrochloride

The flupirtine free base (2.03 g, 6.67 mmol) of Example 3b was dissolved in 92 mL of diethyl ether with the aid of sonication. The solution was stirred under a blanket of nitrogen gas and treated drop wise over approximately one minute with 0.576 mL of 37% hydrochloric acid (7.0 mmol of HCl). Precipitation occurred immediately. The resulting slurry was stirred overnight and filtered through qualitative filter paper. The wet solids were dried in a vacuum oven (22° C., ˜30 in. Hg vacuum) for approximately 7 hours (to a constant weight, as indicated by <0.36% weight loss between weighings) to give 2.13 g (6.24 mmol, 93% yield) of solid flupirtine HCl.

### Example 4c

### Preparation of Flupirtine Hydrochloride

A mixture of 2.5 g (8.2 mmol) of the flupirtine free base of Example 3c and 440 mL of water were heated in a 3-L round-bottom flask with a heating mantle, treated with 8.7 mL of 1 N HCl (8.7 mmol) and stirred at about 64° C. After approximately 2 hours 200 mL of water was added. After stirring for another 2 hours 28 mL of ethanol were added. The mixture was stirred at about 60-64° C. for another 2 hours, the heater was turned off, and the mixture was gently stirred for approximately 18 hours. The sample was rotary evaporated over about 6 hours, utilizing a 45° C. water bath until remaining liquid barely covered resulting solid. The mixture was vacuum filtered and the filter cake was washed with five 20-mL portions of water which were first used to rinse the flask. The solid was left on the filter under vacuum for about 15 minutes, then dried under a stream of nitrogen gas for about 15 hours to give 577 mg (21% yield) of solid flupirtine HCl.

### Example 5

### Preparation and Characterization of 1:1 Flupirtine Hydrochloride Maleic Acid Cocrystal

### Example 5.1.a

### Preparation of 1:1 Flupirtine Hydrochloride Maleic Acid Cocrystal

A mixture of 7.4 g of maleic acid and 75 mL of acetonitrile was vigorously shaken for approximately 10 minutes at ambient temperature and the resulting supernatant was filtered through a 0.22-μm Teflon disc. A 50-mL aliquot of the filtrate was added to 634 mg (1.86 mmol) of the flupirtine hydrochloride of Example 4a and the resulting mixture was agitated overnight at ambient temperature, during which time solids crystallized. The mixture was filtered through qualitative filter paper and the recovered solids were dried in a vacuum oven (23-24° C., ˜30 in. Hg vacuum) for approximately 12 hours to a constant weight, indicated by less than 0.11% weight loss between weighings, to give 551 mg (1.21 mmol, 65% yield) of the 1:1 flupirtine hydrochloride maleic acid cocrystal.

### Example 5.1.b

A mixture of 873 mg of maleic acid and 15 mL of acetonitrile was mixed on a rotating wheel for approximately 2 hours and the resulting supernatant was filtered through a 0.22-μm Teflon disc. To 10 mL of the filtrate was added 104 mg (0.305 mmol) of the flupirtine hydrochloride of Example 4b. The resulting slurry was agitated on a rotating wheel for approximately 4 days and filtered through a 0.22 micrometer nylon filter membrane inside a Millipore Swinnex filter body. The recovered solid was dried in a vacuum oven at ambient temperature for about 1 hour to give 110 mg (79% yield) of the 1:1 flupirtine hydrochloride maleic acid cocrystal.

### Example 5.1.c

A saturated solution of maleic acid in acetonitrile was prepared by agitating a slurry of 164 mg of maleic acid and 3.5 mL of acetonitrile on a rotating wheel for about 30 min and filtering the slurry to remove solid material. To 2 mL of the filtrate was added 10.5 mg of the flupirtine HCl of Example 4c and the resulting slurry was agitated on a rotating wheel for approximately 2 days, during which time all the solid dissolved to give a clear solution. An additional 10.2 mg of the flupirtine HCl of Example 4c was added and the resulting slurry was agitated on a rotating wheel for approximately 7 days, after which time undissolved solid remained. The solid was recovered by filtration of the slurry through a 0.22 micrometer nylon filter membrane inside a Millipore Swinnex filter body and dried briefly under a stream of nitrogen. XRPD analysis indicated that the solid was the 1:1 flupirtine hydrochloride maleic acid cocrystal.

### Example 5.2

### Characterization of 1:1 Flupirtine Hydrochloride Maleic Acid Cocrystal

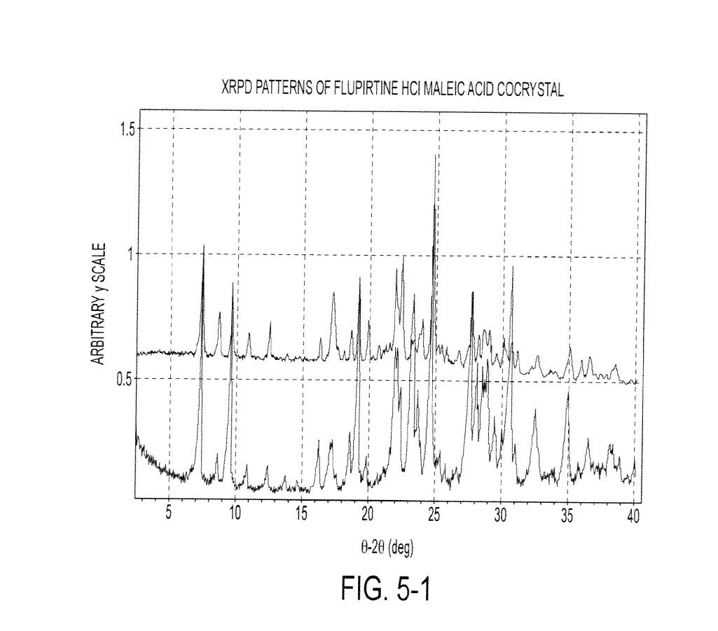

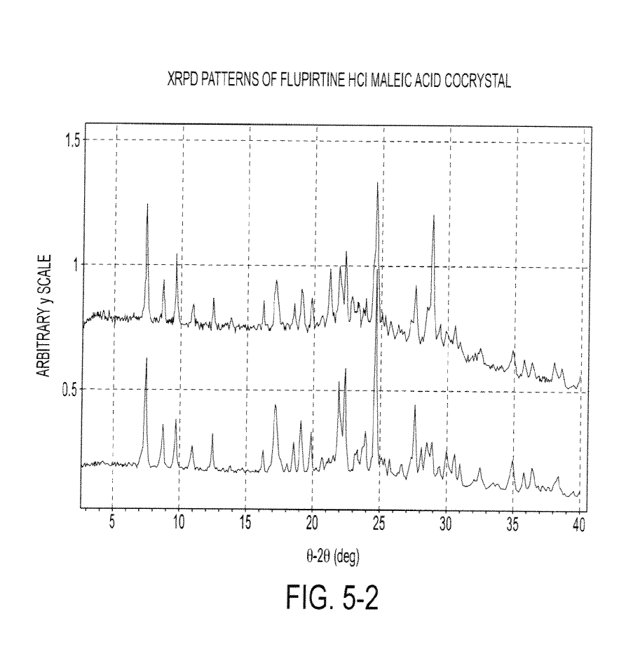

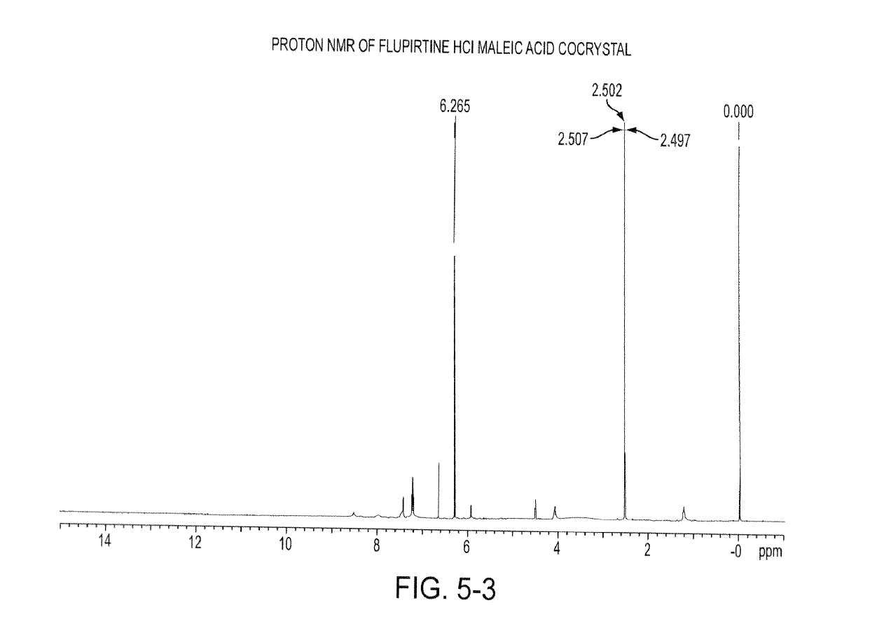

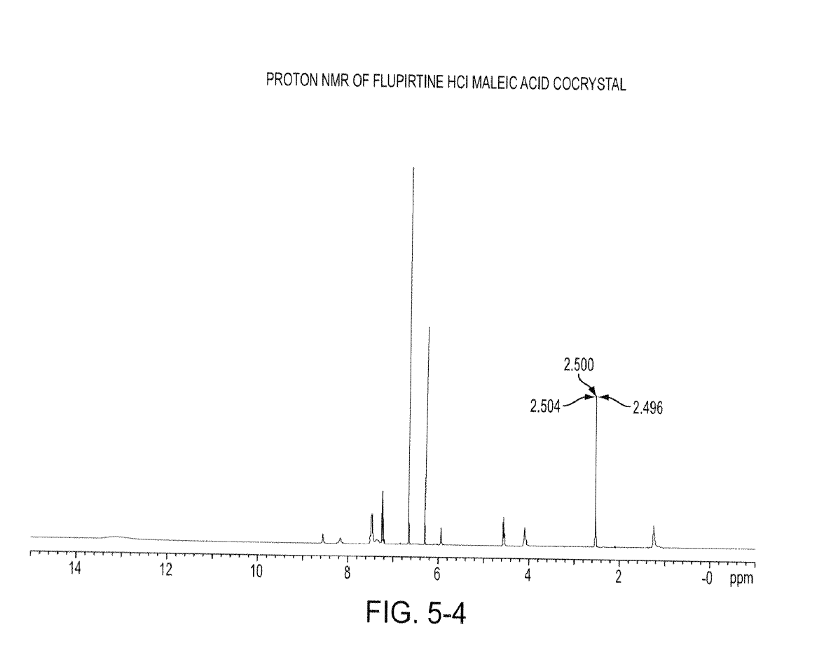

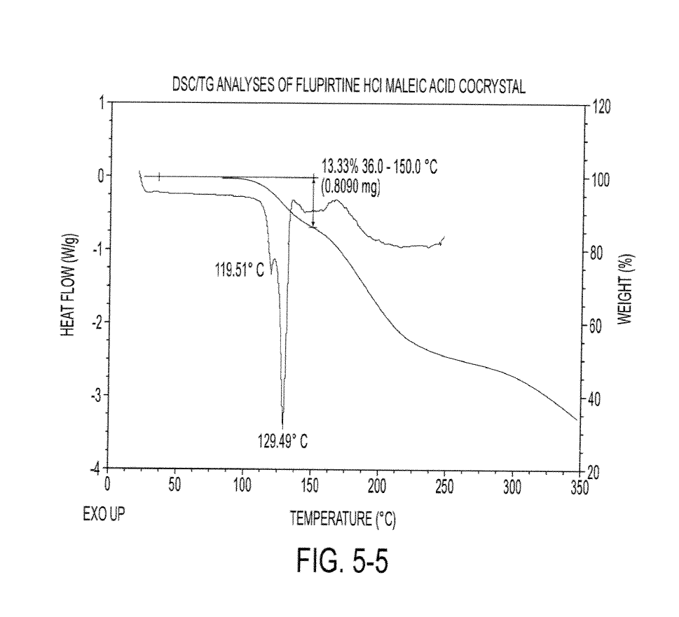

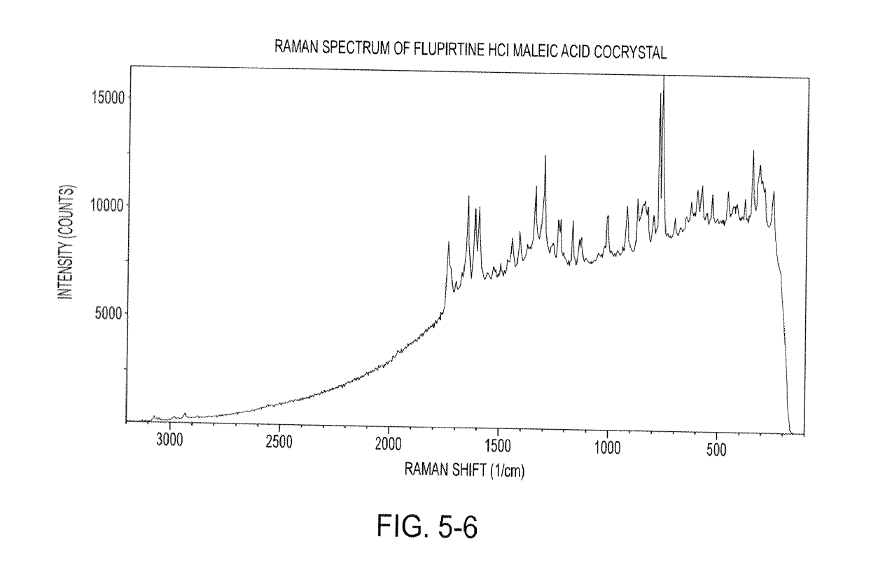

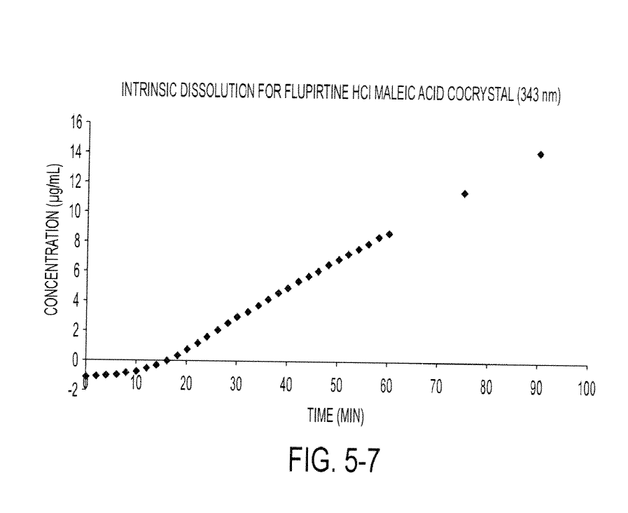

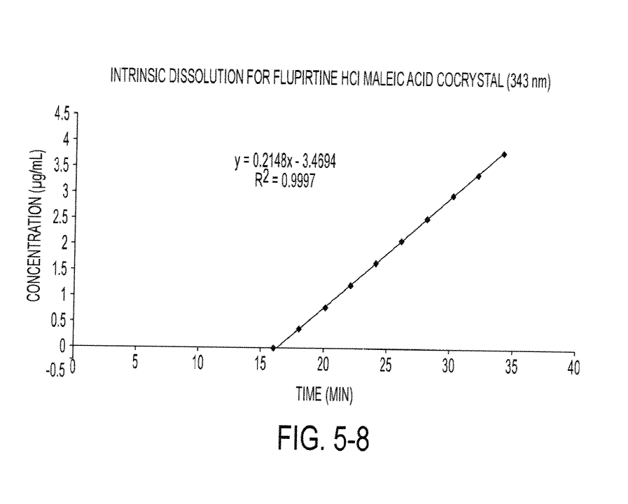

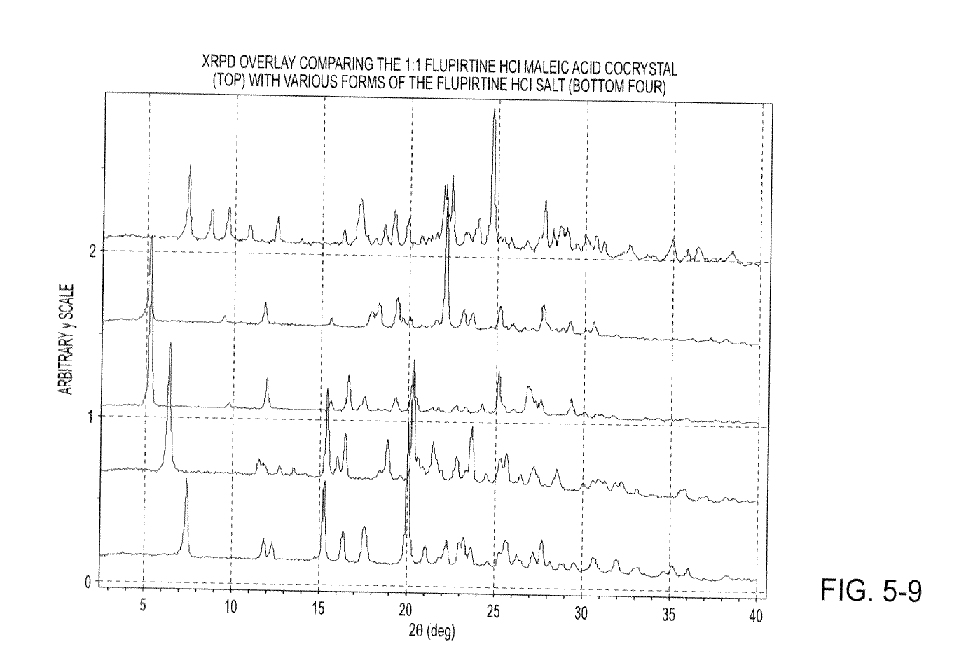

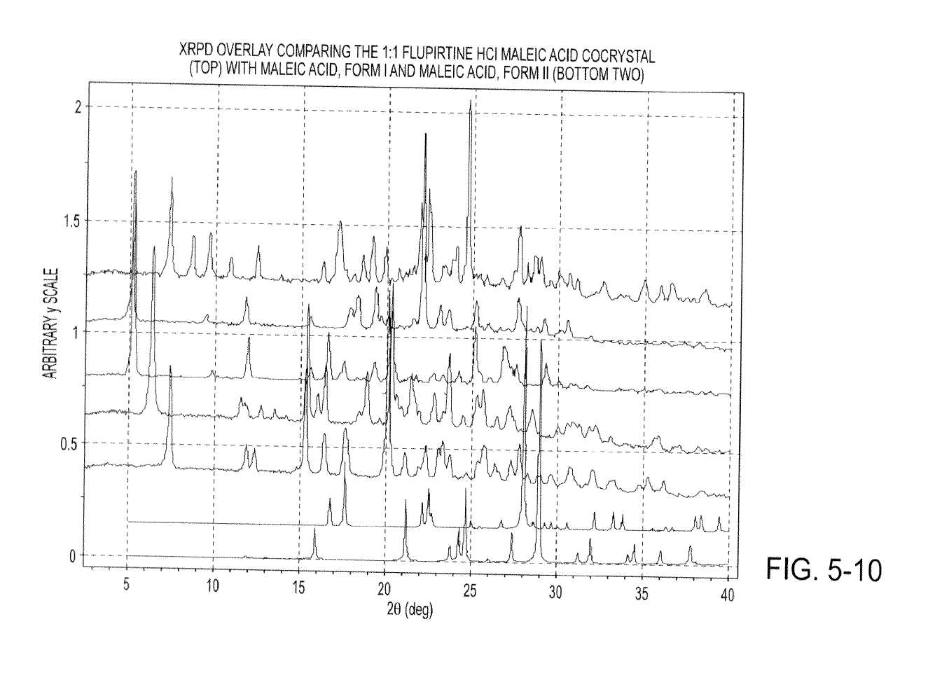

Samples of the 1:1 flupirtine hydrochloride maleic acid cocrystals from Examples 5.1.a and 5.1.c were characterized by XRPD. FIG. 5-1 depicts two XRPD patterns of the 1:1 flupirtine hydrochloride maleic acid cocrystal from Example 5.1.a. The top XRPD was run on an Inel XRG-3000 Diffractometer and the bottom XRPD was run on a Shimadzu XRD-6000 Diffractometer. FIG. 5-2 depicts the XRPD pattern of the 1:1 flupirtine hydrochloride maleic acid cocrystal from Example 5.1.c (top) and Example 5.1.a (bottom), both run on an Inel XRG-3000 Diffractometer.

The XRPD patterns depicted in FIG. 5-1 are similar in terms of the positions (in °2θ) of the peaks, but the relative intensities of the peaks differ between the patterns. The same sample was used for both analyses, so the difference in the appearance of the patterns likely results from preferred orientation, a common phenomenon that affects XRPD patterns. As understood by those of skill in the art, preferred orientation results from the alignment of crystals having anisotropic habits (such as needles or plates) as they are prepared for analysis. The resulting non-random presentation of the crystals to the x-ray beam causes certain crystal planes to be under- or over-represented to the beam, resulting in the peaks arising from those planes to be lower or higher in intensity than they would be if the crystals in the sample were oriented randomly.

The XRPD patterns depicted in FIG. 5-2 are similar in terms of both the positions (in °2θ) and relative intensities of the peaks. A different sample was used for each analysis, so the similarity of the patterns indicates that each sample is the same crystalline form, in this case the 1:1 flupirtine hydrochloride maleic acid cocrystal. A close examination of the XRPD patterns in FIG. 5-2 reveals that there are peaks present in each individual pattern that are not present in the other pattern. For example, a peak at 21.1°2θ is present in the XRPD pattern of the 1:1 flupirtine hydrochloride maleic acid cocrystal from Example 5.1.c (top) and a peak at 28.0°2θ is present in the XRPD pattern of the 1:1 flupirtine hydrochloride maleic acid cocrystal from Example 5.1.a (bottom). A comparison of these XRPD patterns to the XRPD patterns of crystalline maleic acid and crystalline fumaric acid (beta crystalline form) suggests that the peak at 21.1°2θ arises from fumaric acid and that the peak at 28.0°2θ arises from maleic acid, which are present as impurities, discussed below.

Table 5-1 reports the peaks identified in the Shimadzu XRPD pattern for Example 5.1.a and Table 5-2 reports the peaks identified in the Inel XRPD pattern for Example 5.1.c.

TABLE 5-1
Peak Positions of the XRPD Pattern (Shimadzu) for 1:1 Flupirtine
Hydrochloride Maleic Acid Cocrystal from 5.1.a

| °2θ | Intensity (%) |
|---|---|
| 7.3 ± 0.2 | 80 |
| 8.6 ± 0.2 | 17 |
| 9.5 ± 0.2 | 85 |
| 10.7 ± 0.2 | 12 |
| 12.3 ± 0.2 | 12 |
| 13.7 ± 0.2 | 6 |
| 14.5 ± 0.2 | 6 |
| 16.1 ± 0.2 | 22 |
| 17.2 ± 0.2 | 19 |
| 18.5 ± 0.2 | 25 |
| 19.1 ± 0.2 | 87 |
| 19.8 ± 0.2 | 15 |
| 21.9 ± 0.2 | 57 |
| 23.2 ± 0.2 | 75 |
| 23.6 ± 0.2 | 40 |
| 24.6 ± 0.2 | 100 |
| 25.3 ± 0.2 | 15 |
| 25.7 ± 0.2 | 13 |
| 26.6 ± 0.2 | 11 |
| 27.5 ± 0.2 | 81 |
| 28.0 ± 0.2 | 52 |
| 28.4 ± 0.2 | 51 |
| 28.8 ± 0.2 | 50 |
| 29.4 ± 0.2 | 29 |

TABLE 5-2
Peak Positions of the XRPD Pattern (Inel) for 1:1 Flupirtine
Hydrochloride Maleic Acid Cocrystal from Example 5.1.c

| °2θ | Intensity (%) |
|---|---|
| 7.3 ± 0.2 | 78 |
| 8.6 ± 0.2 | 29 |
| 9.6 ± 0.2 | 46 |
| 10.8 ± 0.2 | 16 |
| 12.4 ± 0.2 | 21 |
| 13.7 ± 0.2 | 9 |
| 16.2 ± 0.2 | 18 |
| 17.1 ± 0.2 | 31 |
| 18.5 ± 0.2 | 18 |
| 19.0 ± 0.2 | 27 |
| 19.8 ± 0.2 | 21 |
| 20.5 ± 0.2 | 10 |
| 21.1 ± 0.2 | 37 |
| 21.8 ± 0.2 | 42 |
| 22.3 ± 0.2 | 50 |
| 22.8 ± 0.2 | 21 |
| 23.3 ± 0.2 | 19 |
| 23.6 ± 0.2 | 16 |
| 23.9 ± 0.2 | 21 |
| 24.6 ± 0.2 | 100 |
| 25.7 ± 0.2 | 9 |
| 26.3 ± 0.2 | 10 |
| 27.3 ± 0.2 | 15 |
| 27.6 ± 0.2 | 38 |
| 28.9 ± 0.2 | 88 |
| 29.5 ± 0.2 | 18 |

FIG. 5-3 depicts the proton NMR spectrum of the 1:1 flupirtine hydrochloride maleic acid cocrystal from Example 5.1.a in DMSO-d₆ and Table 5-3 lists the peaks in that spectrum. The presence of a peak at about 6.6 ppm indicates that there is fumaric acid in the sample. The amount of fumaric acid in the sample is estimated by the NMR integrations to be about 5 weight percent. The formation of the fumaric acid likely arises by isomerization of maleic acid in the presence of an organic base (flupirtine), a process that has been observed previously under similarly mild conditions for both maleic acid and its esters (see Clemo and Graham, J. Chem. Soc., 1930, 213-216; Chatterjee S et al, Tetrahedron Lett., 1998, 39: 2843-2846; and WO2003049688).

In Table 5-3 it is shown that the number of olefinic protons indicated by the integral of the peak at 6.26 ppm is about 2.5, which is more than the number expected (2) for the 1:1 ratio of flupirtine hydrochloride to maleic acid present in the cocrystal. It is probable that the high integral results from unreacted maleic acid in the sample. The presence of maleic acid is consistent with the observation of a peak in the XRPD pattern thought to arise from crystalline maleic acid.

FIG. 5-4 depicts the proton NMR spectrum of the 1:1 flupirtine hydrochloride maleic acid cocrystal from Example 5.1.c in DMSO-d₆ and the peaks are listed in Table 5-4. The presence of a peak at about 6.6 ppm indicates that there is fumaric acid in the sample. The amount of fumaric acid in the sample is estimated by the NMR integrations to be about 30 weight percent. The presence of fumaric acid is consistent with the observation of peaks in the XRPD pattern thought to arise from crystalline fumaric acid. In Table 5-4 it is shown that the number of olefinic protons indicated by the integral of the peak at 6.26 ppm is about 2, which is consistent with the analyzed solid being 1:1 flupirtine hydrochloride maleic acid cocrystal.

Together, the two NMR spectra are consistent with the samples from Example 5.1.a and Example 5.1.c being the 1:1 flupirtine hydrochloride maleic acid cocrystal.

TABLE 5-3
¹H NMR Peak Positions for FIG. 5-3

| | peak | | coupling | |
|---|---|---|---|---|
| | position | | constant | number |
| protons | (ppm) | Multiplicity | (Hz) | of protons |
| CH₃ | 1.21 | broad triplet | ~5 | 3 |
| exchangeable | 3.47 | broad singlet | — | — |
| proton(s) | | | | |
| CH₂O | 4.05 | Quartet | 7 | integral obscured |
| | | | | by exchangeable |
| | | | | proton(s) |
| CH₂N | 4.48 | Doublet | 4 | integral obscured |
| | | | | by exchangeable |
| | | | | proton(s) |
| pyridine 5-H | 5.90 | Doublet | 9 | 1 |
| CH═CH | 6.26 | Singlet | — | 2.5 |
| fumaric acid | 6.63 | Singlet | — | — |
| impurity | | | | |
| aromatic | 7.17-7.23 | Multiplet | — | integral obscured |
| | | | | by exchangeable |
| | | | | proton(s) |
| aromatic | 7.41-7.47 | Multiplet | — | integral obscured |
| | | | | by exchangeable |
| | | | | proton(s) |
| exchangeable | 7.97 | broad singlet | — | — |
| proton(s) | | | | |
| exchangeable | 8.51 | broad singlet | — | — |
| proton(s) | | | | |
| exchangeable | 12.70 | broad singlet | — | — |
| proton(s) | | | | |

TABLE 5-4
¹H NMR Peak Positions for FIG. 5-4

| | peak | | coupling | |
|---|---|---|---|---|
| | position | | constant | number |
| protons | (ppm) | Multiplicity | (Hz) | of protons |
| CH₃ | 1.21 | broad triplet | 6 | 3 |
| exchangeable | ~3.6 | broad singlet | — | — |
| proton(s) | | | | |
| CH₂O | 4.05 | Quartet | 7 | integral obscured |
| | | | | by exchangeable |
| | | | | proton(s) |
| CH₂N | 4.51 | Doublet | 4 | 2 |
| pyridine 5-H | 5.91 | Doublet | 9 | 1 |
| CH═CH | 6.27 | Singlet | — | 2 |
| fumaric acid | 6.63 | Singlet | — | — |
| impurity | | | | |
| aromatic | 7.18-7.23 | Multiplet | — | 2 |
| exchangeable | 7.32 | broad singlet | — | — |
| proton(s) | | | | |
| aromatic | 7.42-7.48 | Multiplet | — | 3 |
| exchangeable | 8.14 | broad singlet | — | — |
| proton(s) | | | | |
| exchangeable | 8.53 | broad singlet | — | — |
| proton(s) | | | | |
| exchangeable | 13.12 | broad singlet | — | — |
| proton(s) | | | | |

FIG. 5-5 depicts the DSC/TGA analyses of the 1:1 flupirtine hydrochloride maleic acid cocrystal from Example 5.1.b. The DSC shows two endothermic peaks at ˜120 and 129° C., with accompanying weight loss indicated by TGA.

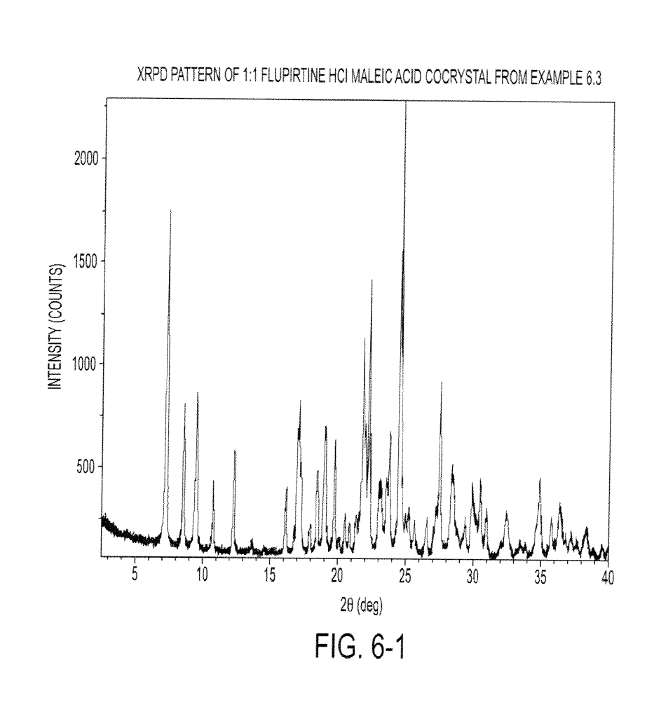

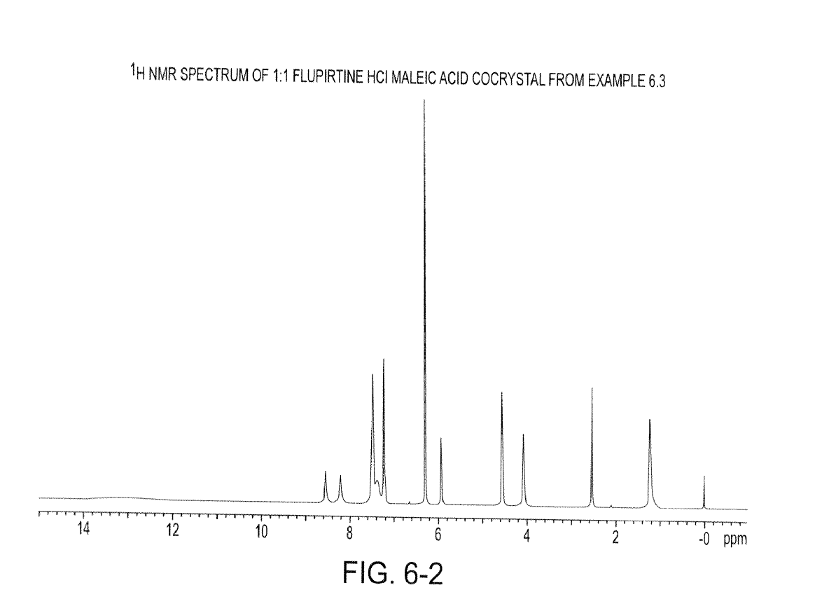

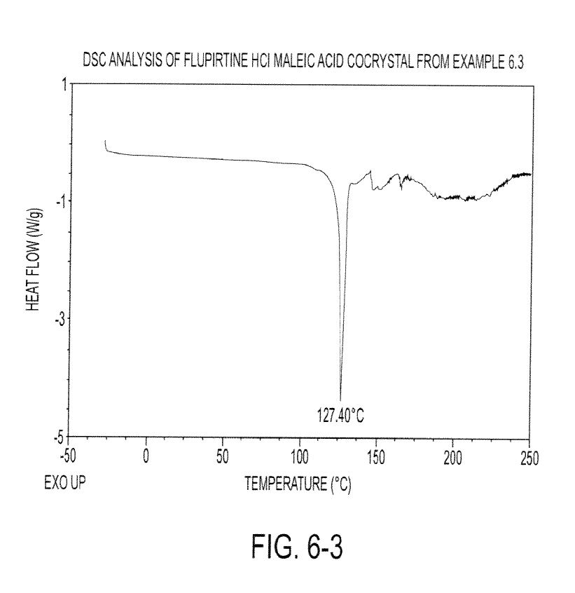

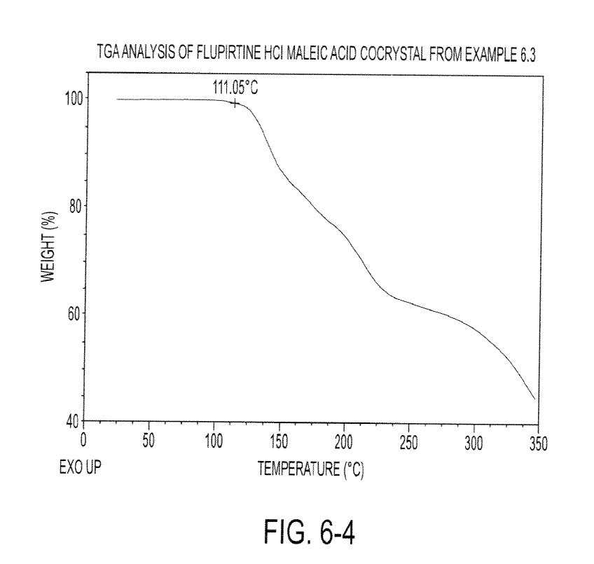

FIG. 5-6 depicts the FT Dispersive-Raman spectrum of the 1:1 flupirtine hydrochloride maleic acid cocrystal from Example 5.1.b, obtained on a Renishaw Mk1 Ramascope model 1000 spectrometer. Table 5-5 reports the absorbance peaks in the Raman spectrum. In its Raman spectrum, 1:1 flupirtine hydrochloride maleic acid cocrystal may be characterized by two or more peaks at the positions listed in the table below.

TABLE 5-5
Peaks in the Raman Spectrum of 1:1 Flupirtine
Hydrochloride Maleic Acid Cocrystal

| Peak position (cm⁻¹) | Intensity |
|---|---|
| 749.6 | 16410 |
| 764.3 | 15630 |
| 338.6 | 13180 |
| 1288 | 12750 |
| 306.1 | 12450 |
| 572.2 | 11460 |
| 1328 | 11320 |
| 591.8 | 11310 |
| 247.8 | 11280 |
| 451.7 | 11200 |
| 523.5 | 11010 |
| 375.2 | 10820 |
| 862.2 | 10810 |
| 1637 | 10750 |
| 827.5 | 10720 |
| 619.6 | 10710 |
| 411.1 | 10600 |
| 9010 | 10500 |
| 1586 | 10360 |
| 1604 | 10250 |
| 788.5 | 10080 |
| 997.5 | 10040 |
| 692.3 | 9934 |
| 1222 | 9843 |
| 1214 | 9845 |
| 1157 | 9784 |
| 1401 | 9239 |
| 1119 | 8981 |
| 1435 | 8943 |
| 1726 | 8713 |

### Example 5.3

### Intrinsic Dissolution

Intrinsic dissolution experiments were performed under sink conditions in water at 37° C. FIG. 5-7 depicts the intrinsic dissolution curve for the 1:1 flupirtine hydrochloride maleic acid cocrystal in water at 37° C., and FIG. 5-8 shows the portion of the data within the Beer's Law plot. The intrinsic dissolution rate of the 1:1 flupirtine hydrochloride maleic acid cocrystal was ˜0.21 μg/mL/min. There appeared to be a slow chemical change occurring in the dissolution medium. The solid recovered from the intrinsic dissolution experiment was analyzed by XRPD and determined to correspond to a mixture of the flupirtine hydrochloride maleic acid cocrystal and a small amount of an unidentified material.

### Example 5.4

### Comparison of XRPD Data

FIG. 5-9 depicts an XRPD overlay comparing the 1:1 flupirtine HCl maleic acid cocrystal (top) with various forms of the flupirtine HCl salt (bottom four). FIG. 5-10 depicts an XRPD overlay comparing the 1:1 flupirtine HCl maleic acid cocrystal (top) with maleic acid, form I (second from bottom) and maleic acid, form II (bottom). Also included in FIG. 5-10 are the four flupirtine HCl salt XRPDs that are included in FIG. 5-9. These two XRPD overlays confirm that the cocrystal is not a physical mixture of the HCl salt and maleic acid.

### Example 6

### Preparation of 1:1 Flupirtine Hydrochloride Maleic Acid Cocrystal by Milling and Its Characterization

### Example 6.1

### Preparation of Flupirtine Free Base

To a 250-mL separatory funnel containing 2.9 g (6.9 mmol) of flupirtine maleate was added 50 mL of diethyl ether. Saturated aqueous sodium bicarbonate (75 mL) was added, and the funnel was shaken vigorously. Additional diethyl ether (75 mL) and saturated aqueous sodium bicarbonate (25 mL) were added with shaking until almost all solids were dissolved. The top, ether layer, was removed and washed twice with 10-mL portions of water. The pH of the final wash water was approximately 5-6 (stick). The ether layer was dried by addition of solid magnesium sulfate. The resulting mixture was filtered through a 0.2-μm nylon filter into a clean glass round bottom flask. A precipitate formed in the filtered solution. Additional diethyl ether (30 mL) was added with stirring and the precipitate dissolved.

### Example 6.2

### Preparation of Flupirtine Hydrochloride

The solution of free base in diethyl ether from Example 6.1 was acidified by addition of 6.9 mL (6.9 mmol) of 1.0 N hydrochloric acid in diethyl ether over a few minutes. Precipitation occurred immediately on acid addition. The flask was covered with aluminum foil and the mixture was stirred overnight. Solids were recovered by vacuum filtration, washed with two 10-mL portions of diethyl ether, and allowed to dry in the filter funnel for several minutes to give 1.9 g (79% yield from flupirtine maleate) of flupirtine hydrochloride. X-ray powder diffraction analysis showed the solids to be a mixture of crystalline forms of flupirtine hydrochloride.

### Example 6.3

### Preparation of 1:1 Flupirtine Hydrochloride Maleic Acid Cocrystal by Milling

To a steel milling jar was added a steel ball, 99 mg 0.29 mmol) of flupirtine hydrochloride from example 6.2, 34 mg (0.29 mmol) of maleic acid, and 10 μL of acetone. The jar was capped, placed in a Retsch MM200 mixer and milled at 30 Hz for 2 minutes. The jar was opened and solids were scraped from the interior surfaces, and another 10 μL of acetone were added. The jar was capped, placed in the Retsch mixer, and milled at 30 Hz for 2 minutes. That procedure was repeated three more times. The solid was removed from the milling jar to give 97 mg (73% yield) of yellow 1:1 flupirtine hydrochloride maleic acid cocrystal.

### Example 6.4

### Characterization of 1:1 Flupirtine Hydrochloride Maleic Acid Cocrystal

A sample of the 1:1 flupirtine hydrochloride maleic acid cocrystal from Example 6.3 was characterized by XRPD using a Panalytical X-Pert Pro diffractomer. FIG. 6-1 depicts the XRPD pattern of that 1:1 flupirtine hydrochloride maleic acid cocrystal, which shows agreement with the XRPD patterns in FIGS. 5-1 and 5-2. Table 6-1 reports the peaks identified in FIG. 6-1.

TABLE 6-1
Peak Positions of the XRPD Pattern (Panalytical) for 1:1 Flupirtine
Hydrochloride Maleic Acid Cocrystal from Example 6.3

| °2θ | Intensity (%) |
|---|---|
| 7.3 ± 0.10 | 73 |
| 8.6 ± 0.10 | 35 |
| 9.5 ± 0.10 | 38 |
| 10.8 ± 0.10 | 16 |
| 12.3 ± 0.10 | 25 |
| 13.7 ± 0.10 | 7 |
| 16.2 ± 0.10 | 17 |
| 17.0 ± 0.10 | 32 |
| 17.1 ± 0.10 | 34 |
| 18.0 ± 0.10 | 10 |
| 18.5 ± 0.10 | 21 |
| 19.1 ± 0.10 | 31 |
| 19.8 ± 0.10 | 28 |
| 20.1 ± 0.10 | 7 |
| 20.6 ± 0.10 | 12 |
| 20.9 ± 0.10 | 10 |
| 21.4 ± 0.10 | 12 |
| 21.9 ± 0.10 | 48 |
| 22.1 ± 0.10 | 32 |
| 22.3 ± 0.10 | 62 |
| 23.2 ± 0.10 | 18 |
| 23.6 ± 0.10 | 20 |
| 23.9 ± 0.10 | 26 |
| 24.6 ± 0.10 | 100 |
| 25.1 ± 0.10 | 12 |
| 25.3 ± 0.10 | 14 |
| 25.7 ± 0.10 | 10 |
| 26.6 ± 0.10 | 10 |
| 27.6 ± 0.10 | 41 |
| 28.4 ± 0.10 | 23 |
| 29.5 ± 0.10 | 11 |

FIG. 6-2 depicts the proton NMR spectrum of the 1:1 flupirtine hydrochloride maleic acid cocrystal from Example 6.3 in DMSO-d₆. The absence of a resonance at 6.6 ppm indicates that the cocrystal prepared in Example 6.3 does not contain fumaric acid. In contrast, the cocrystals prepared in Examples 5.1.a and 5.1.c contain some fumaric acid, as evident from the presence of a resonance at 6.6 ppm in their NMR spectra (FIGS. 5-3 and 5-4, respectively). Table 6-12 lists the peaks in the ¹H NMR of FIG. 6-2.

TABLE 6-12
¹H NMR Peak Positions for FIG. 6-2.

| | peak | | coupling | |
|---|---|---|---|---|
| | position | | constant | number |
| Protons | (ppm) | multiplicity | (Hz) | of protons |
| CH₃ | 1.21 | broad multiplet | — | 3 |
| CH₂O | 4.05 | broad quartet | 7 | 2 |
| CH₂N | 4.54 | broad singlet | — | 2 |
| pyridine 5-H | 5.92 | doublet | 9 | 1 |
| CH═CH | 6.27 | Singlet | — | 2 |
| (maleic acid) | | | | |
| aromatic | 7.18-7.22 | multiplet | — | integral |
| | | | | obscured by |
| | | | | exchangeable |
| | | | | proton(s) |
| exchangeable | 7.36 | broad singlet | — | — |
| proton(s) | | | | |
| aromatic | 7.42-7.46 | multiplet | — | integral |
| | | | | obscured by |
| | | | | exchangeable |
| | | | | proton(s) |
| exchangeable | 8.19 | broad singlet | — | — |
| proton(s) | | | | |
| exchangeable | 8.52 | broad singlet | — | — |
| proton(s) | | | | |
| exchangeable | 13.04 | broad singlet | — | — |
| proton(s) | | | | |

The DSC thermogram, FIG. 6-3, of the 1:1 flupirtine hydrochloride maleic acid cocrystal prepared in Example 6.3 exhibits a sharp endotherm at ˜127° C., which corresponds to the weight loss observed in the TGA thermogram, FIG. 6-4. Heating the cocrystal to ˜150° C. by TGA followed by XRPD analysis of the recovered solids showed the solids to be flupirtine hydrochloride, indicating loss of maleic acid from the cocrystal.

1. A 1:1 2-amino-3-carbethoxyamino-6-(p-fluorobenzylamino)pyridine hydrochloride (flupirtine hydrochloride) maleic acid cocrystal.

2. A 1:1 2-amino-3-carbethoxyamino-6-(p-fluorobenzylamino)pyridine hydrochloride (flupirtine hydrochloride) maleic acid cocrystal characterized by a powder x-ray diffraction pattern having two or more peaks at 7.3°2θ±0.2°2θ; 8.6°2θ±0.2°2θ; 9.6°2θ±0.2°2θ; 10.8°2θ±0.2°2θ; 12.4°2θ±0.2°2θ; 13.7°2θ±0.2°2θ; and 16.2°2θ±0.2°2θ.

3. A pharmaceutical composition for treating a nervous system disorder, a pain disorder, or a musculoskeletal disorder, comprising a therapeutically effective amount of a cocrystal of claim 1 and a pharmaceutically acceptable carrier.

4. The pharmaceutical composition of claim 3, further comprising an analgesic selected from the group consisting of strong and weak opioids, NSAIDs, COX-2 inhibitors, acetaminophen, anti-inflammatories, tricyclic antidepressants, anticonvulsant agents, voltage gated calcium channel blockers, N-type calcium channel blockers, calcium channel modulators, SNRIs, monoamine reuptake inhibitors, sodium channel blockers, NMDA antagonists, AMPA antagonists, glutamate modulators, GABA modulators, CRMP-2 modulators, NK-1 antagonists, TRPV1 agonists, cannabinoids, adenosine agonists, nicotinic agonists, p38 MAP kinase inhibitors, corticosteroids, and combinations thereof.

5. A method for treating a central nervous system disorder in a mammal, comprising administering to a patient in need thereof a therapeutically effective amount of a cocrystal of claim 1.

6. The method of claim 5, wherein the central nervous system disorder is selected from the group consisting of epilepsy, Creutzfeldt-Jakob Disease, Alzheimer's Disease, Parkinson's Disease, Huntington's Disease, Batten Disease, cerebral ischemia, schizophrenia, psychosis, mood disorders, major depressive disorder, dysthymia, anxiety disorders, overactive bladder, urinary incontinence, urinary flow problems as a result of prostate hyperplasia, irritable bowel syndrome, and tinnitus.

7. A method for treating pain, comprising administering to a patient in need thereof a therapeutically effective amount of a cocrystal of claim 1.

8. The method of claim 7, wherein the pain is selected from the group consisting of back pain, neck pain, pain resulting from traumatic injury, post-operative pain, post-dental procedure pain, dysmenorrhea, osteoarthritis, visceral pain, cancer pain, rheumatoid arthritis, psoriatic arthritis, gout, tendonitis pain, bursitis pain, musculoskeletal pain, sports injury-related pain, sprains, strains, pain of osteoporosis, ankylosing spondylitis, migraine, tension headache, temporomandibular joint pain, fibromyalgia, myofascial pain syndrome, pain of irritable bowel syndrome, interstitial cystitis, and idiopathic chronic pain.

9. The method of claim 7, wherein the pain is an acute or chronic neuropathic pain or pain associated with a nervous system disorder.

10. The method of claim 9, wherein the acute or chronic neuropathic pain or pain associated with a nervous system disorder is selected from the group consisting of painful diabetic neuropathy, postherpetic neuralgia, trigeminal neuralgia, complex regional pain syndrome I, complex regional pain syndrome II, ischemic neuropathy, phantom limb pain, chemotherapy-induced neuropathy, HIV-related neuropathy, AIDS-related neuropathy, neuropathic back pain, neuropathic neck pain, carpal tunnel syndrome, other forms of nerve entrapment or nerve compression pain, brachial plexus lesions, other peripheral nerve lesions, neuropathic cancer pain, vulvodynia, central neuropathic pain, pain due to multiple sclerosis, post-stroke pain, Parkinson's Disease related central pain, postoperative chronic pain, Guillain-Barre syndrome (GBS), Charcot-Marie-Tooth (CMT) disease, idiopathic peripheral neuropathy, alcoholic neuropathy,

11. The method of claim 7, wherein the pain is an acute or chronic condition of a pathological muscle contracture.

12. The method of claim 11, wherein the acute or chronic condition of a pathological muscle contracture is selected from the group consisting of discomfort, muscle spasm, stiffness, back pain, neck pain, neck-shoulder-arm syndrome, scapulohumeral periarthritis, cervical spondylosis, spasticity or spastic paralysis of neurological origin due to multiple sclerosis, spinal cord injury, traumatic brain injury, cerebral palsy, stroke or cerebrovascular disorder, spastic spinal paralysis, sequelae of surgical trauma, amyotrophic lateral sclerosis, spinocerebellar degeneration, spinal vascular disorders, subacute myelo-optico neuropathy (SMON), primary dystonia, secondary dystonia, and muscle cramps.

13. A method for treating diabetes mellitus or a neurodegenerative disease of the nervous or visual system resulting in a complication of the diabetes, comprising administering to a patient in need thereof a therapeutically effective amount of a cocrystal of claim 1.

14. The method of claim 13, wherein the neurodegenerative disease of the nervous or visual system resulting in a complication of the diabetes is selected from the group consisting of diabetic neuropathy, diabetic retinopathy, diabetic maculopathy, glaucoma, diabetic gastroparesis, cataracts, and foot ulcers.

15. A method of making a 1:1 2-amino-3-carbethoxyamino-6-(p-fluorobenzylamino)pyridine hydrochloride (flupirtine hydrochloride) maleic acid cocrystal comprising the step of:
milling flupirtine hydrochloride and maleic acid in acetone.
